(19) United States
(12) Patent Application Publication
(10) Pub. No.: US 2018/0168457 A1
(43) Pub. Date: Jun. 21, 2018

(54) HOLDING MEMBER AND HOLDING MEMBER SET, MASKING MEMBER AND MASKING MEMBER SET, AND PHOTOACOUSTIC APPARATUS

(71) Applicant: CANON KABUSHIKI KAISHA, Tokyo (JP)

(72) Inventor: Hisafumi Ebisawa, Tokyo (JP)

(21) Appl. No.: 15/838,459

(22) Filed: Dec. 12, 2017

(30) Foreign Application Priority Data
Dec. 19, 2016 (JP) 2016-245348
Nov. 24, 2017 (JP) 2017-225660

(51) Int. Cl. A61B 5/00; G01N 21/17; A61B 8/06; A61B 5/1455

(52) U.S. Cl. CPC A61B 5/0095; G01N 21/1702; A61B 2576/00; A61B 5/14551; A61B 8/06

(57) ABSTRACT

A photoacoustic apparatus is used that includes a light irradiating unit irradiating a subject that is part of an examinee with light; a probe receiving an acoustic wave generated from the subject irradiated with the light and outputs a signal; and a determining unit determining, based on information related to a specific light absorbing region on a surface of the subject, whether or not a masking member that optically masks the specific light absorbing region is arranged at a specific position.

### BACKGROUND OF THE INVENTION

### Field of the Invention

The present invention relates to a holding member that holds a subject and a holding member set, a masking member that is used in the holding member and a masking member set, and a photoacoustic apparatus that includes the holding member or the masking member.

### Description of the Related Art

In recent years, in the medical field and the like, a study of an apparatus that uses photoacoustic imaging is in progress. The photoacoustic imaging is a technique in which pulsed light is applied to a subject, an acoustic wave (photoacoustic wave) generated from an absorber that has absorbed energy of the light having propagated and diffused in the subject is detected, and characteristic information related to an optical characteristic value of the inside of the subject is visualized. As a three-dimensional visualization technique that uses the photoacoustic imaging, there is a technique called photoacoustic tomography in which the photoacoustic wave generated from the absorber is detected using a probe, and three-dimensional data related to the optical characteristic value is produced by performing image reconstruction calculation. For example, by using light having a wavelength that allows absorption by hemoglobin as the pulsed light, it is possible to image a blood vessel image in the subject noninvasively. In addition, depending on the wavelength to be used, it is also possible to image collagen and elastin under the skin. Further, by using a contrast medium, it is also possible to enhance the blood vessel image and image a lymph vessel.

In the case where the subject is a living body, a part that contains a large amount of pigment such as a mole or a hair that is present on the skin or in the outer layer of the skin, or a nipple or an areola in a breast has a light absorption amount larger than those of the other parts, and hence the intensity of the photoacoustic wave generated when light is applied thereto is higher than those of the other parts. When the photoacoustic wave generated from such a subject is imaged, the influence of the photoacoustic wave based on the mole or the hair becomes significant, and an artifact that depends on the surface tissue having high absorbance occurs in an acquired image. On the other hand, there is a possibility that characteristic information in a dermic layer or a deep part of the living body that is present under the surface tissue having high absorbance is not imaged appropriately, and it is not possible to appropriately perform evaluation in image diagnosis.

To cope with such a problem, Japanese Patent Application Laid-open No. 2016-053482 discloses that the surface tissue (specific part) having high absorbance such as the mole or the hair in the subject is detected and the detected specific part is trimmed from an area in which light irradiation is performed or an area in which detection of the photoacoustic wave is performed. With this, the influence of the photoacoustic wave generated from the specific part is reduced.

Patent Literature 1: Japanese Patent Application Laid-open No. 2016-053482

### SUMMARY OF THE INVENTION

In the case of the method described in Japanese Patent Application Laid-open No. 2016-053482 in which the irradiation region is trimmed in a software manner, output fluctuations of a light source have sometimes occurred before and after the region trimming. In addition, in the method described in Japanese Patent Application Laid-open No. 2016-053482 in which a reception signal used at the time of reconstruction is masked in a software manner, there are cases where an error occurs in estimation of a propagation path, and the quality of a reconstructed image has sometimes been reduced.

When a wide area including the surrounding area of the specific part is not subjected to the light irradiation and the acoustic wave detection, it is possible to suppress the influence of the specific part. However, in this case, a region in which photoacoustic measurement cannot be performed is increased. In addition, it is also possible to reduce the influence of the specific part by not utilizing a strong signal from the specific part at the time of image reconstruction.

However, the light irradiation to the specific part is not limited in this method, and hence the influence of a signal deriving from the specific part is exerted on a wide area of the subject so that the ratio of the signal that is not used is increased and the quality of the image is reduced.

The present invention has been made in view of the above problem. A subject of the present invention is to provide a technique capable of acquiring a photoacoustic image in which the artifact is reduced even in the case where the absorber having the large light absorption amount is present on the surface of the subject of the photoacoustic measurement.

The present invention provides a photoacoustic apparatus comprising:

a light irradiating unit configured to irradiate a subject that is part of an examinee with light;

a probe configured to receive an acoustic wave generated from the subject irradiated with the light and output a signal; and

a determining unit configured to determine, based on information related to a specific light absorbing region on a surface of the subject, whether or not a masking member that optically masks the specific light absorbing region is arranged at a specific position.

In addition, the present invention provides a holding member set, including a plurality of holding members which holds a subject and is selectively used for a photoacoustic apparatus, the photoacoustic apparatus receives and images photoacoustic waves generated from the subject held by the holding member, wherein

the holding member set is configured to have the plurality of holding members in which masking members that are different in at least any of position, size, and shape are arranged correspondingly to a specific light absorbing region on a surface of the subject.

Further, the present invention provides a masking member set, including a plurality of masking members which is to be arranged on a specific position of a holding member and is selectively used for a photoacoustic apparatus, the photoacoustic apparatus receives and images photoacoustic waves generated from a subject held by the holding member, wherein

the masking member set includes the plurality of masking members that are different in at least one of shape and size from each other.

According to the present invention, it is possible to provide the technique capable of acquiring the photoacoustic image in which the artifact is reduced even in the case where the absorber having the large light absorption amount is present on the surface of the subject of the photoacoustic measurement.

Further features of the present invention will become apparent from the following description of exemplary embodiments with reference to the attached drawings.

### BRIEF DESCRIPTION OF THE DRAWINGS

FIGS. 1A and 1B are views showing the configuration of a photoacoustic apparatus in a first embodiment;

FIGS. 2A and 2B are views showing details of image processing when a region is specified;

FIGS. 3A and 3B are views showing an example of a masking member;

FIG. 4 is a view showing diffracted light around the masking member;

FIG. 5 is a flowchart for explaining processes in the first embodiment;

FIGS. 6A and 6B are views for explaining a holding member in a second embodiment;

FIG. 7 is a view showing the configuration of the photoacoustic apparatus in a third embodiment;

FIG. 8 is a flowchart for explaining processes in the third embodiment;

FIG. 9 is a view showing an example of the masking member in the third embodiment;

FIGS. 10A to 10D are views showing the state of bonding of the masking member to the holding member; and

FIGS. 11A to 11C are views showing the configuration of the holding member in the third embodiment.

### DESCRIPTION OF THE EMBODIMENTS

Hereinbelow, with reference to the drawings, preferred embodiments of the present invention will be described. Note that the dimensions, materials, shapes, and relative arrangements of components described below should be appropriately changed according to the configuration of an apparatus to which the invention is applied and various conditions. Therefore, the scope of the invention is not limited to the description below.

The present invention relates to a technique for detecting an acoustic wave propagating from a subject, and producing and acquiring characteristic information of the inside of the subject. Therefore, the present invention is viewed as a subject information acquiring apparatus or a control method thereof, an acoustic wave receiving apparatus or a control method thereof, or a subject information acquiring method or a signal processing method. The present invention is also viewed as a program that causes an information processing apparatus including hardware resources such as a CPU and a memory to execute these methods, or a non-transitory computer-readable storage medium in which the program is stored.

The subject information acquiring apparatus of the present invention includes a photoacoustic imaging apparatus that receives an acoustic wave that is generated inside a subject by a photoacoustic effect when light (electromagnetic wave) is applied to the subject, and acquires characteristic information of the subject. In this case, the characteristic information is information on a characteristic value that is produced by using a reception signal deriving from the received photoacoustic wave and corresponds to each of a plurality of positions inside the subject.

The characteristic information acquired by photoacoustic measurement is a value in which an absorption amount and absorptance of light energy are reflected. The characteristic information includes, e.g., the generation source of the photoacoustic wave generated by light irradiation having a single wavelength, an initial sound pressure in the subject, or a light energy absorption density or an absorption coefficient derived from the initial sound pressure. In addition, from the characteristic information obtained by using a plurality of mutually different wavelengths, it is possible to acquire the concentration of a substance that constitutes a tissue. By determining oxygenated hemoglobin concentration and deoxygenated hemoglobin concentration as the substance concentration, it is possible to calculate an oxygen saturation distribution. In addition, as the substance concentration, glucose concentration, collagen concentration, melanin concentration, and volume fraction of fat or water are also determined.

A two-dimensional or three-dimensional characteristic information distribution is obtained based on the characteristic information of each position inside the subject. Distribution data can be produced as image data. As the characteristic information, instead of the numerical data, distribution information of each position inside the subject may also be determined. That is, examples of the distribution information include an initial sound pressure distribution, an energy absorption density distribution, an absorption coefficient distribution, and an oxygen saturation distribution.

The acoustic wave mentioned in the present invention is typically an ultrasonic wave, and includes an elastic wave called a sound wave or an acoustic wave. An electrical signal converted from the acoustic wave by a transducer, an acoustic transducer element or the like is also referred to as an acoustic signal. Note that the description of the ultrasonic wave or the acoustic wave in the present specification is not intended to limit the wavelength of the elastic wave thereof. The acoustic wave generated by the photoacoustic effect is also referred to as a photoacoustic wave or a photo-ultrasonic wave. An electrical signal deriving from the photoacoustic wave is also referred to as a photoacoustic signal.

Each of the following embodiments will describe a photoacoustic apparatus that acquires the distribution of a light absorber inside a subject by applying pulsed light to the subject, receiving the acoustic wave from the subject using the photoacoustic effect, and analyzing the acoustic wave. Such a photoacoustic apparatus is suitably used for diagnosis of blood vessel diseases, malignant tumors or the like of a human and an animal and a follow-up of chemotherapy. Examples of the subject include part of a living body such as a breast or a hand of an examinee, an animal other than a man such as a mouse, an inanimate subject, and a phantom.

### First Embodiment

System Outline

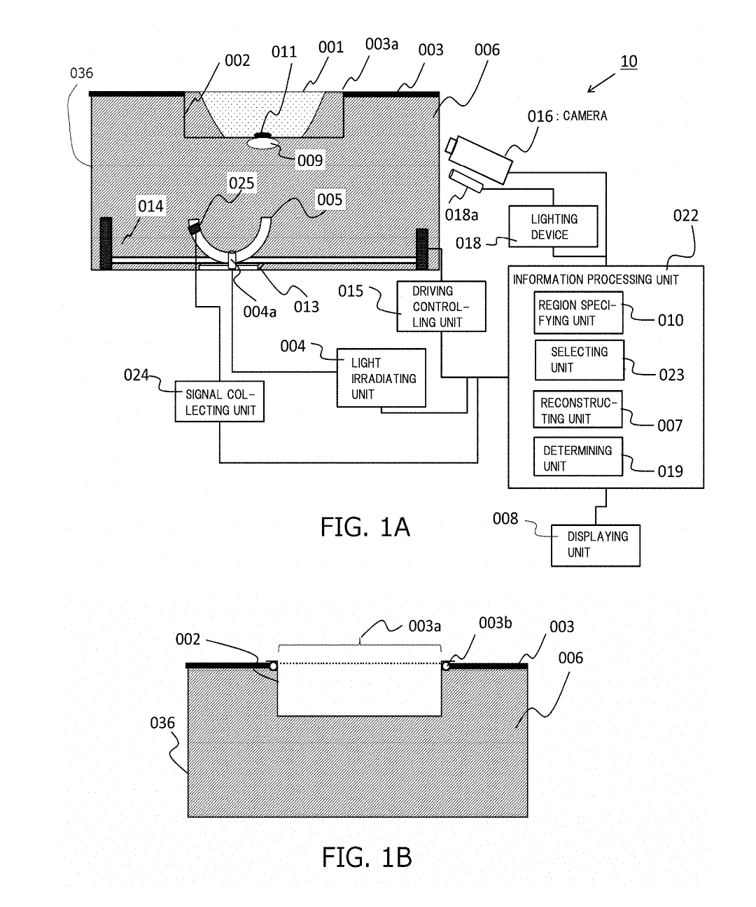

FIG. 1A is a schematic view of a system of a photoacoustic apparatus 10 of the present embodiment. FIG. 1B is a schematic view of a system of a photoacoustic apparatus 10 of the present embodiment. A subject 001 serving as a measurement target is a breast. The photoacoustic apparatus 10 includes a holding member 002 that holds the subject 001, a supporting base 003 to which the holding member 002 is fixed and that supports an examinee, and a light irradiating unit 004 that irradiates the subject 001 with light via the holding member 002. The photoacoustic apparatus further includes a probe 005 that receives the photoacoustic wave emitted from the subject 001 irradiated with measurement light, and a signal collecting unit 024 that collects an electrical signal outputted from the probe. The photoacoustic apparatus further includes a vessel 0036 configured to store an acoustic matching liquid 006 that allows the acoustic wave to propagate between the holding member 002 and the probe 005. The photoacoustic apparatus 10 further includes an image reconstructing unit 007 that produces a characteristic information distribution from an electrical signal outputted from the signal collecting unit 024 and converts the characteristic information distribution into image data, and a displaying unit 008 that displays an image. An information processing unit 022 includes the image reconstructing unit 007, a region specifying unit 010, a selecting unit 023, and a determining unit 019.

A masking member 009 optically masks light from the light irradiating unit 004. The region specifying unit 010 specifies the position and the size of a specific light absorbing region 011 that is a tissue having high absorbance positioned in the vicinity of the surface of a mole, a hair, a nipple, an areola or the like positioned in a region of the subject 001 serving as the measurement target. The selecting unit 023 selects the masking member 009 suitable for the specific light absorbing region based on the position and the size of the specific light absorbing region 011 obtained from the region specifying unit 010. In addition, the selecting unit 023 selects the appropriate holding member 002 from the group of a plurality of the holding members 002 having regions of the masking members 009 that are different in at least position or size. That is, in other words, the selecting unit 023 is a mask selecting unit that selects the masking member 009 suitable for a specific position, or a holding member selecting unit that selects the holding member 002 provided with the masking member 009 suitable for the specific position.

The determining unit 019 determines whether or not the masking member 009 is arranged at the specific position. In other words, the determining unit 019 determines whether or not the holding member 002 has the masking member 009 at the specific position. A driving mechanism 014 and a driving controlling unit 015 change relative positions of the probe, the subject, and the holding member. Note that at least any of the signal collecting unit 024, the image reconstructing unit 007, the displaying unit 008, the driving mechanism 014, the light irradiating unit 004, the driving controlling unit 015, and the information processing unit 022 is configured such that the operation thereof is changed or suspended based on the determination result of the determining unit 019.

In addition, the specific position mentioned above is located between the specific light absorbing region 011 and the light irradiating unit 004, and is preferably located on the side of the specific light absorbing region 011 between the specific light absorbing region 011 and the light irradiating unit 004 from the viewpoint of reducing the influence of diffracted light of the masking member 009.

Function and Preferable Configuration of Each Component

Holding Member and Supporting Base

The holding member 002 holds and fixes the subject 001 to stabilize the shape of the subject 001. With this, computational accuracy in calculation of delay time and signal processing at the time of image reconstruction is improved. As the holding member 002, a member that transmits the acoustic wave and light is used. In addition, a member having high stiffness or a member having stretchability is preferable so as to be able to hold the subject 001. Examples of the member having high stiffness include resin materials such as polyethylene terephthalate (PET), polymethyl pentene, and acrylic. Examples of the material having stretchability include a rubber sheet made of latex or silicone and a material such as urethane. In addition, the holding member 002 obtained by combining a plurality of materials may also be used. In the present embodiment, the holding member 002 having a thickness of not more than 1 mm and made of PET is used.

The optimum shape and material of the holding member 002 differ depending on the part and the size of the subject 001. In addition, there are cases where replacement of the holding member 002 is desired from the viewpoint of sanitation. In view of the above points, it is preferable to have a plurality of interchangeable holding members 002 having different shapes and sizes. In the example in FIGS. 1A and 1B, In order to mount the holding member 002 interchangeably, it is preferable to provide an attaching mechanism 003b such as mounting hardware or a hook that can easily fix the holding member 002 in the periphery of an opening portion 003a of the supporting base 003. In other words, the attaching mechanism 003b is called as an installation portion 003b for installation of the holding member 002. The installation portion 003b is located around the insertion opening 003a configure to be installed a holding member. As the material of the supporting base 003, a material having strength that is high enough to support the weight of the examinee such as, e.g., stainless is used. In the present embodiment, the supporting base 003 is positioned above a mechanism that performs photoacoustic measurement, and constitutes an upper surface of a bed on which the examinee lies facedown. The examinee inserts a left or right breast from the opening portion 003a.

Light Irradiating Unit

The light irradiating unit 004 is a mechanism that irradiates the subject 001 with pulsed light, and includes a laser light source, an optical member for light transmission, and an irradiation opening 004a serving as an emission end of light. The light source applies pulsed laser light having a pulse interval of 10 Hz to the subject 001. As the light source, it is possible to use a solid laser such as a titanium sapphire laser, a gas laser, a dye laser, and a semiconductor laser. In addition, other than the laser, it is possible to use a flash lamp and a light-emitting diode. As the irradiation light, near-infrared light is preferable. The wavelength of light is preferably about 650 to 1100 nm, and is set to 750 nm in the present embodiment. Note that, in order to determine the constituent concentration and oxygen saturation of the subject 001, it is preferable to use a wavelength-variable laser capable of emitting lights having a plurality of wavelengths. Light is guided from the light source to the irradiation opening 004a by optical members such as a bundle fiber, a lens, a mirror, and a prism.

Note that the light irradiation by the light irradiating unit 004 is not limited to the pulsed light and, as long as irradiation intensity temporally changes such that an irradiated region thermally expands or contracts, the light irradiation does not necessarily need to include a non-irradiation period. The light irradiating unit 004 is configured to perform the light irradiation in the case where the masking member 009 is arranged at the specific position, and is configured not to perform the light irradiation in the case where the masking member 009 is not arranged at the specific position based on the determination result related to the presence or absence of the masking member 009 by the determining unit 019. In other words, the photoacoustic measurement is started based on the determination result of the determining unit 019.

In addition, in the case where the masking member 009 is not arranged at the specific position, the determining unit 019 displays information indicating that “the masking member is not arranged appropriately” in the displaying unit 008, and presents the information to an operator. In addition, the determining unit 019 outputs information indicating whether or not the optical masking member 009 is used in photographing based on an input command of the operator.

Subject and Specific Light Absorbing Region

Herein, the subject 001 serving as the measurement target will be described. When a light absorber present inside the subject 001 or on the surface thereof absorbs energy of irradiation light, the acoustic wave is generated by thermal expansion. The light absorber having an absorption characteristic that allows absorption of near-infrared light includes blood in a living body that contains a large amount of hemoglobin and melanin. A large amount of melanin is contained in each of the mole and the hair in the vicinity of the outer layer of the skin and the nipple of the breast. On the other hand, particularly in the photoacoustic measurement performed for the purpose of image diagnosis of breast cancer, in consideration of the fact that a cancer tissue includes a large number of neovascular vessels, it is necessary to image a blood vessel tissue in a deep part (e.g., about 2 to 3 cm in depth from the surface) in the breast. However, the photoacoustic wave generated from melanin that is present on the surface of the subject and has a large light absorption amount hides the photoacoustic wave generated from a blood vessel in the deep part of the subject, and obstructs acquisition of an accurate photoacoustic image.

In the following description, the absorber that is present on the surface of the subject or in the vicinity thereof and has relatively high absorbance such as the mole, the hair, the nipple, or the areola is referred to as the specific light absorbing region 011. A distinction between the specific light absorbing region 011 and the other region is not strict, and the distinction is appropriately made according to the average absorbance of the skin of the examinee and the purpose of the measurement. The area of the specific light absorbing region 011 may be automatically determined by an image recognition process, or may also be determined based on an instruction from a user interface (inputting unit) such as a mouse or a touch pen of the information processing unit 022. In the case where the instruction is received from the inputting unit, the operator may be allowed to specify the specific light absorbing region, or may also be allowed to specify a brightness threshold value for determination of the specific light absorbing region 011.

Acoustic Matching Liquid

The acoustic wave generated from the subject 001 by the light irradiation passes through the holding member 002 and the acoustic matching liquid 006, and is received by a receiving element 025 of the probe 005. The acoustic matching liquid 006 acoustically matches the subject 001 (or the holding member 002) to the probe 005. As the acoustic matching liquid 006, an acoustic matching liquid that allows the acoustic wave to propagate and does not obstruct scanning of the probe 005 is preferable. Examples of such an acoustic matching liquid include liquids such as water, diisodecyl sebacate (DIDS), polyethylene glycol (PEG), silicone oil, and castor oil. In the present embodiment, water is used. Note that the acoustic matching liquid 006 may also be provided between the holding member 002 and the subject 001.

Probe and Receiving Element

The receiving element 025 of the probe 005 receives the acoustic wave, converts the acoustic wave to an analog electrical signal, and outputs the analog electrical signal. As the receiving element 025, it is possible to use lead zirconate titanate (PZT), polyvinylidene fluoride (PVDF), and capacitive micro-machined ultrasonic transducers (cMUT). In addition, it is also possible to use a Fabry-Perot probe. An improvement in SN ratio and a reduction in measurement time are supposed to be achieved by using the probe 005 in which a plurality of the receiving elements are arranged one-dimensionally, two-dimensionally, on a curved surface, or on a spherical surface. In the present embodiment, there is used the probe 005 in which several hundred receiving elements 025 made of PZT each having a center frequency of 2 MHz and an area of 1 sq. mm are arranged on an inner surface of a hemispherical supporting member. FIG. 1 shows only one receiving element 025 for the sake of simplicity. When this probe 005 is used, it is possible to form a high-sensitivity region in which reception directivity fluxes of a plurality of the receiving elements 025 are concentrated in the vicinity of the center of curvature of the hemisphere, and image the subject with high quality. In addition, as the probe 005, an array probe in which several hundred receiving elements made of PZT similar to the above receiving elements are arranged two-dimensionally is also preferable.

The signal collecting unit 024 performs an amplification process and a digital conversion process on the electrical signal outputted by the receiving element 025. The signal collecting unit 024 is constituted by combining a reception amplifier, an A/D converter, and a correction circuit.

In this example, the probe 005 is disposed below the holding member 002 partially dipped in the vessel 036 storing the acoustic matching liquid 006, and moves in a horizontal plane by the driving mechanism 014 described later. However, the holding mechanism of the acoustic matching liquid is not limited thereto. For example, the inside of the hemispherical supporting body of the probe 005 may be filled with the acoustic matching liquid 006, and the holding member 002 may be soaked in the acoustic matching liquid 006.

Driving Mechanism

The driving mechanism 014 moves the probe 005 and the irradiation opening 004a mounted on a carriage 013 in a specific measurement region. The driving controlling unit 015 controls the operation of the driving mechanism 014. The carriage 013 scans a two-dimensional plane opposing the subject 001. As the driving mechanism 014, it is possible to use, e.g., a combination of a pulse motor and a ball screw, and a linear motor. In addition, the driving mechanism 014 may drive the probe 005 in three-dimensional directions. The driving mechanism 014 allows acquisition of an image from a wide area and an improvement in the quality of a reconstructed image by an increase in reception signal area. Note that the driving mechanism 014 is not essential. As the driving controlling unit 015, an information processing apparatus shared by the information processing unit 022 described later may be used, or an independent physical configuration may be provided.

Image Reconstructing Unit

In the image reconstructing unit 007 of the information processing unit 022, the characteristic information distribution is produced based on a digital electrical signal deriving from the photoacoustic wave. At this point, the image reconstructing unit 007 determines the delay time of the electrical signal based on coordinate information of the position of each receiving element 025, performs a delay process on each electrical signal, and then reconstructs the image. In the image reconstruction process and the delay time determination, a process for acquiring a digital signal stored in a memory is performed based on a distance between each receiving element 025 and the position of a target (a pixel or a voxel), and the acoustic velocity of a propagation substance of the acoustic wave.

It is possible to use any known method in the image reconstruction. Examples of the method include a back projection method, a phasing addition method, and a Fourier transform method. Note that, at the time of the reconstruction, the image may be reconstructed for each signal acquired at each scanning position of the probe 005, but it is preferable to store the signal acquired at each scanning position in a memory, and then collectively use the group of the signals for the reconstruction. With this, an improvement in SN ratio and an improvement in the quality of the image resulting from an increase in viewing angle are supposed to be achieved.

Region Specifying Unit

The region specifying unit 010 of the information processing unit 022 will be described. The region specifying unit 010 analyzes surface information of the subject 001 acquired by a camera 016, and calculates and determines the area of the specific light absorbing region 011. First, the camera 016 acquires a surface image of the subject 001 placed in the holding member 002, and transmits the surface image to the region specifying unit 010. As the camera 016, it is possible to use existing optical imaging apparatuses. The region specifying unit 010 analyzes brightness information of the surface image, and sets a region having a value that is not more than a preset threshold value as the specific light absorbing region 011.

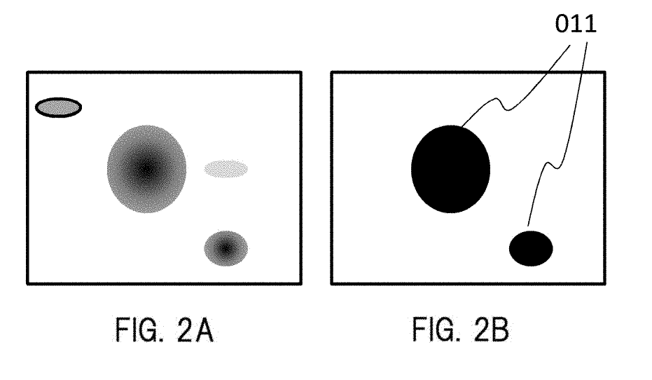

FIG. 2A shows the brightness information of the surface image. FIG. 2B shows a situation in which the region specifying unit 010 sets the area of the specific light absorbing region 011. In addition to the brightness information, it is also possible to analyze color information to set the specific light absorbing region 011. Specific light absorbing components of the mole, the nipple, and the hair that are considered to be factors of the artifact in the present invention absorb infrared light well, and hence, when blue and green components are identified in image processing, it is easy to determine the specific light absorbing region 011. Thus, the method that refers to the color information has an advantage that, even in the case where, e.g., a red operation marker is used in the subject 001, the marker is less likely to be recognized as the specific light absorbing region 011.

When the specific light absorbing region 011 is determined using the threshold value of the brightness or the color, it is preferable to reduce the influence of indoor lighting, and irradiate the subject 001 using a lighting device of which the light amount and characteristics are known. Accordingly, in the present embodiment, a material that blocks the indoor lighting is used as the material of the supporting base 003 or the wall of the vessel 0036 of the acoustic matching liquid 006. In addition, light is applied to the subject 001 by using a lighting device 018 that emits known light. In an example in FIG. 1, the lighting device 018 is another light source different from the light irradiating unit 004. The lighting device 018 does not need to be a laser. The lighting device 018 may be an LED or an incandescent lamp, and is preferably capable of emitting white light. Note that the light irradiating unit 004 may be used as the lighting device 018 without providing the lighting device 018. The lighting device 018 is disposed for the purpose of securing illuminance on the surface of the subject 001 such that the intensity of reflected light from the subject 001 allows the camera 016 to detect the surface condition of the subject and allows the region specifying unit 010 to analyze the surface condition thereof. The wavelength of the lighting device 018 may be any wavelength as long as the wavelength allows the camera 016 to detect the surface condition of the subject, and visible light or infrared light is selected. As the light emitted by the lighting device 018, pulsed light that emits light at a frequency lower than the frame frequency of the camera 016 or DC light that emits light continuously is selected.

With this configuration, the positional relationship among an irradiation opening 018a of the lighting device, the camera 016, and the holding member 002 is determined, and the light amount, the spectrum, and optical imaging conditions of the camera 016 are determined. As a result, it becomes possible to stably analyze the reflectivity and the tinge of the surface of the subject with high reproducibility, and hence accuracy when the masking member 009 is selected is improved.

Note that the region specifying unit 010 can be configured to also serve as the determining unit 019, and the region specifying unit 010 can determine whether or not the masking member 009 is arranged at the specific position.

In addition, the region specifying unit 010 can be configured to be capable of infrared imaging of the subject in a wavelength range including infrared light or near-infrared light, and allow displaying of the specific light absorbing region or the masking member in the displaying unit.

In the case where the holding member 002 is a thin rubber sheet and the specific light absorbing region 011 is a large part such as the nipple, the specific light absorbing region 011 sometimes has a protruding shape. In this case, by moving the camera 016 and acquiring images at a plurality of positions, it is possible to specify the three-dimensional specific light absorbing region 011 with high accuracy.

Selecting Unit

The selecting unit 023 (holding member selecting unit) of the information processing unit 022 selects a given holding member 002 from a set 2000 of a plurality of the holding members 002 based on the position and the size of the specific light absorbing region 011 determined by the region specifying unit 010. In the individual holding members 002, the masking members 009 that are different in at least one of position and size are arranged. Note that the selecting unit 023 can present information related to the selected holding member 002 or masking member 009 in the displaying unit 008.

Information Processing Unit

The information processing unit 022 includes computational resources such as a CPU and a memory, and can be implemented by an information processing apparatus such as a PC or a workstation that operates according to a program or an instruction input. In the example in FIG. 1, the region specifying unit 010, the selecting unit 023, and the image reconstructing unit 007 are configured as functional modules of the information processing unit 022. Note that each functional block may have an independent physical configuration. In addition, the driving controlling unit 015 may also be part of the information processing unit 022.

Displaying Unit

The characteristic information distribution produced in the image reconstructing unit 007 is sent to the displaying unit 008 in the form of image data, and is displayed visually. As the displaying unit 008, it is possible to use any image displaying apparatus such as a liquid crystal display, a cathode-ray tube, a plasma display, or an organic EL display. Note that the photoacoustic apparatus does not need to include the displaying unit 008, and may cause an external display to display the image data produced in the image reconstructing unit 007. The subject of the present invention can be implemented with a photoacoustic apparatus that is not the one that produces the image data. The photoacoustic apparatus may receive and store the photoacoustic wave in which the influence of the absorber on the surface of the subject is reduced, and may output the photoacoustic wave to another information processing apparatus.

Masking Member

The masking member 009 is a member that is arranged on the side of the light irradiating unit 004 of the specific light absorbing region 011 such that the amount of light emitted from the light irradiating unit 004 and reaching the specific light absorbing region 011 is reduced and the acoustic wave generated from the specific light absorbing region 011 is reduced.

The masking member 009 includes a material that reflects or scatters at least part of the irradiation light from the light irradiating unit 004, and has a backside masking function of reducing an intensity of the light transmitted through the masking member 009 in opposition to the side of the light irradiating unit 004 (hereinafter referred to as a backside). The backside masking effect is rephrased into a downstream masking effect, because the masking portion masks a downstream side region in the subject with respect to the masking portion in a light irradiation direction.

In addition, the masking member 009 includes a material that reflects or scatters at least part of the irradiation light from the light irradiating unit 004, and has a self-masking function of reducing light absorption of the masking member 009 by itself. In other words, the masking member 009 has at least two unique functions as the backside masking function for masking a photoacoustic sound source located backside of the masking member 009 in where the specific light absorbing region is included and the self-masking function for masking the masking member 009 as a photoacoustic sound source, configured not to generate a photoacoustic wave by itself.

The masking member 009 reduce the generation of a high-intensity photoacoustic wave due to the specific light absorbing region with the backside masking function and the self-masking function. The high-intensity photoacoustic wave due to the specific light absorbing region likely to disturb that the receiving element 025 receive a weak photoacoustic wave generated around the masking member 009 and the specific light absorbing region 011 due to a low light absorbance and a low intensity of the light attenuated along a light path in the subject. As a result, the masking member 009 allows the probe 005 to receive the weak photoacoustic wave without being disturbed by the high intensity photoacoustic wave due to the specific light absorbing region 011.

As a result, the high-intensity photoacoustic wave deriving from the specific light absorbing region that obstructs a weak photoacoustic signal generated from a living tissue around the specific light absorbing region is reduced, whereby an acoustic wave noise is reduced and the artifact is reduced.

Note that, in the case where a material that absorbs light is used in the masking member instead of the material that reflects or scatters light, i.e., in the case where the masking member without the self-masking function is used, light that reaches the specific light absorbing region 011 is reduced, but the acoustic wave is generated from the masking member. Therefore, as a member that covers the specific light absorbing region 011, a member that has high reflectivity and low absorptivity to measurement light is preferable. Further, the masking member 009 preferably has high transmissivity for the acoustic wave such that the photoacoustic wave generated in the specific light absorbing region positioned on the backside of the masking member 009 is not blocked by the masking member 009 itself. Note that the masking member without the self-masking function includes a tape having the same color as that of the skin and a material having an absorption spectrum similar to that of the subject, has only the backside masking function as the masking function, and is not adequate.

Herein, conditions required for the masking member 009 to have the masking functions will be described algebraically. When spectral transmittance and spectral reflectivity corresponding to a center wavelength λ of light emitted from the light irradiating unit 004 are T(λ) and R(λ) respectively, it is preferable for the masking member 009 to satisfy the following general expression (1). Note that the left side of the general expression (1) corresponds to spectral absorptivity, and the spectral reflectivity T(λ) includes a spectral scattering rate.

[Expression 1]

1−(T(λ)+R(λ))<R(λ) (1)

In addition, it is further preferable for the masking member 009 to have an optical characteristic that satisfies the following general expression (2).

[Expression 2]

T(λ)<R(λ) (2)

Further, it is further preferable for the masking member to have an optical characteristic that satisfies the following general expression (3).

[Expression 3]

1≥T(λ)+R(λ)≤0.75 (3)

The light transmittance of the masking member 009 preferably has a value such that the intensity of the acoustic wave generated from the specific light absorbing region 011 is not more than a predetermined intensity. The predetermined intensity is the average sound pressure of the acoustic wave generated from a tissue (e.g., a blood vessel) other than the specific light absorbing region 011 that is present in the vicinity of the surface of the subject 001 (e.g., within 1 mm in depth from the surface) and is not shielded by the masking member 009.

The state in which the masking member 009 is arranged on the side of the light irradiating unit 004 of the specific light absorbing region and covers the specific light absorbing region is, i.e., the state in which the masking member 009 optically masks the specific light absorbing region.

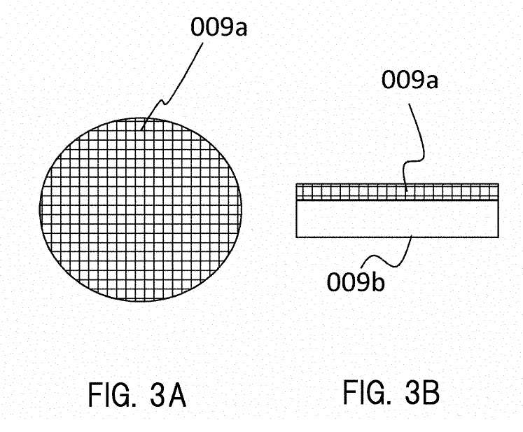

FIG. 3A shows the circular masking member 009 used in the present embodiment. As shown in a cross-sectional view in FIG. 3B, the masking member 009 is obtained by depositing a thin film 009a made of gold on a substrate 009b that is a flat PET plate having a thickness of 150 μm. As the substrate 009b, a resin plate made of acrylic or polymethyl pentene may be used. As the thin film 009a, a metal film made of gold, silver, copper, or aluminum that reflects infrared light well is preferable. In addition, a metal thin plate may also be used as the masking member 009.

A method of reflection of light by the masking member 009 may be a method of a diffusing surface in which the transmittance is reduced by diffuse reflection in addition to a method of a metal film in which passage of light is prevented by specular reflection (regular reflection). As the diffuse reflection-type masking member 009, it is possible to use a diffusion sheet having a fine uneven surface shape, and a sheet mixed with a substance such as titanium oxide that scatters light from the irradiation light source. In the case where the sheet is used, when the thickness of the sheet material is reduced, the acoustic transmittance is improved and image quality is improved. Therefore, it is appropriate to reduce the thickness of the sheet material as much as possible while balancing strength and thickness. In addition, by using a rubber or gel sheet having an acoustic characteristic similar to that of the acoustic matching liquid 006, it is possible to reduce acoustic transmittance and reflection of the acoustic wave on the surface of the sheet, and hence image degradation is reduced. As the material of the sheet, it is possible to use urethane or the like in the case of gel, and it is possible to use natural rubber, isoprene rubber, urethane rubber, silicon rubber, or the like in the case of rubber.

Note that, by using the diffuse reflection-type masking member as the masking member 009, flare of light received by the camera 016 at the time of optical imaging of the surface of the subject 001 of the region specifying unit 010 is less likely to occur. With this, accuracy in the determination of the specific light absorbing region 011 in the region specifying unit 010 is improved.

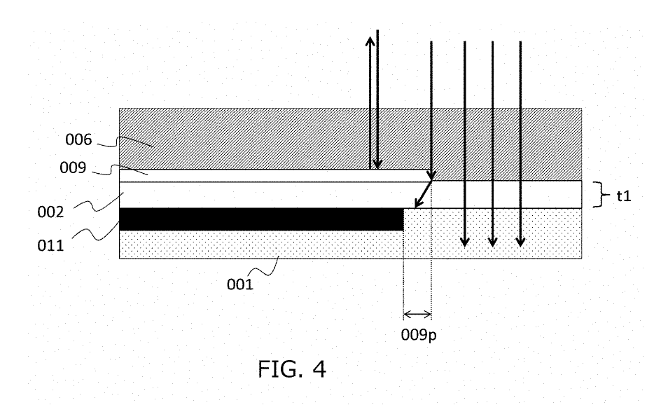

The size of the masking member 009 is preferably moderately larger than the size of the specific light absorbing region 011. The reasons therefor will be described below. First, the reasons include the influence of diffracted light that bends around the end of the masking member 009. The details thereof are shown in FIG. 4. The holding member 002 having a thickness t1 is present between the masking member 009 and the subject 001. The measurement light applied to a region corresponding to the masking member 009 is reflected by the masking member 009, but part of the light bends around the end of the masking member 009 as the diffracted light. The diffracted light progresses in the holding member 002 toward the inside of the region covered by the masking member 009. Accordingly, it is preferable to select the masking member 009 larger in area than the specific light absorbing region 011 such that the light having bent around the end thereof is not applied to the specific light absorbing region 011.

The size of the masking member 009 that is made larger than that of the specific light absorbing region 011 is calculated from the incident angle of light on the masking member, the wavelength of light, the optical characteristic and the thickness of the holding member 002, and the optical characteristic of the acoustic matching liquid 006. For example, consideration is given to the case where the light source having a wavelength of 750 nm and the holding member 002 having the PET thickness t1=1 mm are used, and water is used as the acoustic matching liquid 006. At this point, as shown in FIG. 4, in the case where light is perpendicularly incident on a masking member 009, the influence of the diffracted light can be almost suppressed when a protrusion amount 009p of the masking member 009 is no less than about 0.1 mm. Even in the case where the incident angle of light is not perpendicular, the protrusion amount 009p of the masking portion 009 is no less than about 1 mm that is associated with the thickness of the holding member 002 is adequate. In other words, the masking portion 009 includes protrusion amount 009p peripherally configured to mask over the specific light absorbing region 011 in a full covering manner.

In the selection of the masking member 009 and the selection of the holding member 002 to which the masking member 009 is mounted, it is preferable to consider movement and displacement after the placement of the subject 001. In particular, in the case where the holding member 002 is a resin sheet or plate, there are cases where, after the placement of the subject 001, the subject 001 slides on the holding member 002, and the position of the specific light absorbing region 011 at the time of the photoacoustic measurement is changed from that at the time of the determination of the specific light absorbing region 011. As a result, there is a possibility that it becomes impossible for the masking member 009 to cover the specific light absorbing region 011. To cope with this, it is preferable to set the size of the masking member 009 to a large size in consideration of a positional displacement amount in advance. The positional displacement amount can be calculated from information related to the subject such as the type and the size and information related to the holding member such as the material. More preferably, the displacement amount may be calculated by an experiment. In the case of the present embodiment in which the subject is the breast and the holding member 002 is formed of PET, based on the experiment, it is verified that the displacement of up to about 1 mm occurs during the photoacoustic measurement. Accordingly, the masking member 009 having the protrusion amount 009p from the specific light absorbing region 011 of at least 1 mm is used.

In the case where the cup-shaped holding member 002 in which the center is deeper than a peripheral part is used, when the subject 001 is a soft tissue such as the breast, the subject 001 tends to gradually move toward the deep central part. Accordingly, in the case where the shape of the holding member 002 is tilted, it is also effective to set the masking member 009 widely in a direction in which the depth is increased.

Note that the displacement having a displacement amount larger than an assumed displacement amount can occur. To cope with this, it is effective to detect the specific light absorbing region 011 using the region specifying unit 010 again after the mounting of the masking member 009. With this, it is possible to check whether or not the specific light absorbing region 011 is shielded by the masking member 009. The timing of the checking is preferably immediately before the measurement or after the end of the measurement. In the case where the specific light absorbing region 011 is detected at the time of the checking immediately before the measurement, an instruction to correct the placement state of the breast to the holding member 002 is outputted to the operator from the user interface. In the case where the specific light absorbing region 011 is detected after the end of the measurement, a message that urges the operator to perform re-measurement and a message indicating that the specific light absorbing region 011 is detected are outputted. In the case of the latter, it is preferable to present the shielding state to the operator such that the operator can determine whether or not the re-measurement is executed. As the presentation method of the massage, any method such as voice or an image is used.

Process Flow

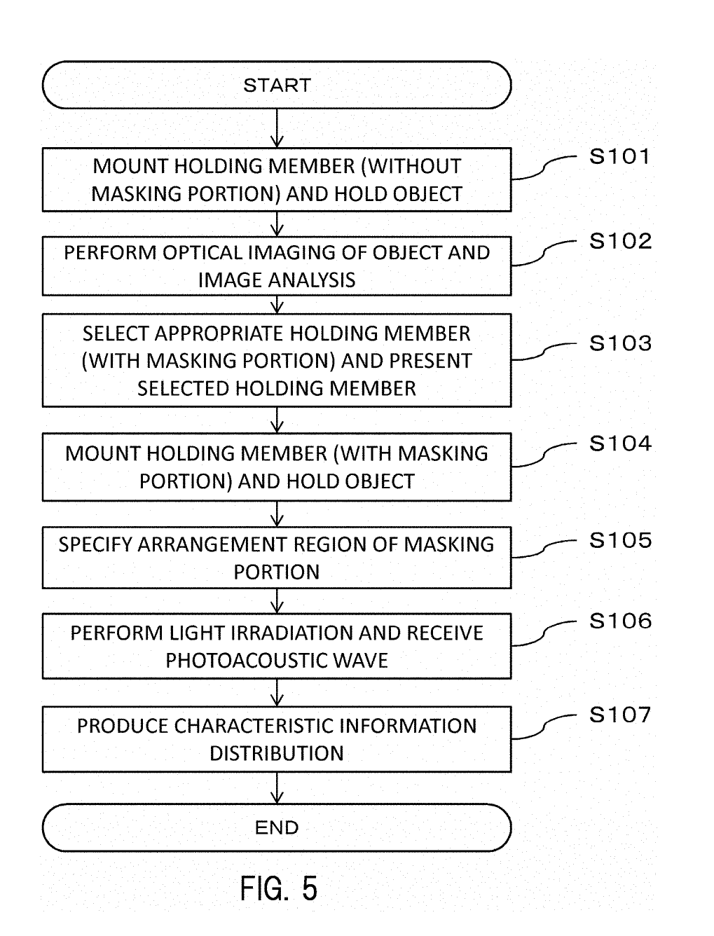

Hereinbelow, with reference to a flowchart in FIG. 5, a series of processes of the present embodiment will be described.

In Step S101, an operator such as a doctor or an engineer places the breast inserted from the opening portion 003a on the holding member 002. Herein, the holding member 002 that is not provided with the masking member 009 is used. In Step S102, the region specifying unit 010 calculates the position and the size of the specific light absorbing region 011 based on a subject surface image acquired by the camera 016 by performing the optical imaging.

In Step S103, the selecting unit 023 selects the holding member 002 that allows the masking member 009 to shield the specific light absorbing region 011 from the irradiation light based on the position and the size of the specific light absorbing region 011. That is, anyone holding member 002 is selected from the holding member set 2000 that includes a plurality of the holding members 002 that are different in the position or size of the masking member 009 from each other. Subsequently, the selecting unit 023 presents the selected holding member 002 to the operator via the displaying unit 008.

In Step S104, the operator holds the breast using the selected holding member 002. At this point, angle adjustment described later may be performed. Next, in Step S105, the region in which the masking member 009 is arranged is specified by the region specifying unit 010 by using the image from the camera 016. In Step S105, further, the specified region in which the masking member 009 is arranged is compared with the specific light absorbing region 011 specified in Step S102, and it is determined whether or not the masking member 009 is arranged at the specific position by the determining unit 019. Next, in Step S106, the light irradiation from the irradiation opening 004a of the light irradiating unit 004 is started based on the information that the masking member 009 is arranged at the specific position in Step S105. Subsequently, the receiving element 025 of the probe 005 receives the photoacoustic wave generated from the breast, converts the photoacoustic wave to the electrical signal, and outputs the electrical signal. At this point, by operating the driving mechanism 014 using the driving controlling unit 015, wide-area measurement is allowed. Next, in Step S107, the image reconstructing unit 007 produces the characteristic information distribution based on the electrical signal. With this, it is possible to acquire the characteristic information distribution indicative of function information of the inside of the subject. In the case where the determination result in Step S105 indicates that the masking member 009 is not arranged at the specific position, photographing operations of the light irradiating unit 004, the probe 005, the driving controlling unit 015, the driving mechanism 014, and the signal collecting unit 024 are stopped in Step S106.

In Step S106, the element of which the operation is changed based on the determination result in Step S105 includes at least any of the signal collecting unit 024, the image reconstructing unit 007, the displaying unit 008, the driving mechanism 014, the light irradiating unit 004, the driving controlling unit 015, and the information processing unit 022.

First Modification of Flow

In Steps S101 to S102 in the flow, after the optical imaging is performed in a state in which the subject 001 is held by the holding member 002 without the masking member 009, the holding member 002 is replaced with the holding member 002 provided with the masking member 009. However, when the replacement operation is performed in this manner, the holding member 002 that is not used in the photoacoustic measurement is required, which leads to increases in cost and labor. To cope with this, the subject 001 may be subjected to the optical imaging and the area of the specific light absorbing region 011 may be determined without using the holding member 002. In this case, with regard to the posture of the examinee, the examinee may lie on the supporting base 003 facedown with the breast inserted into the opening portion 003a similarly to the posture at the time of the actual photoacoustic measurement, and the examinee may also be apart from the supporting base 003. However, in the case where the optical imaging is performed with any posture, the subject 001 that is not fixed by the holding member 002 is imaged, and hence the position and the size of the specific light absorbing region 011 are sometimes changed after the subject 001 is fixed by the holding member 002. In particular, in the case of the breast, the shape of the breast easily changes due to gravity and the influence of an external force. To cope with this, the region specifying unit 010 may appropriately simulate the change of the specific light absorbing region 011 after the subject 001 is held by the holding member 002.

Second Modification of Flow

In Step S103 in the flow, there are cases where information that can be used for the holding member 002 and the masking member 009 is obtained from previous data of the photoacoustic measurement of the examinee (subject 001). In these cases, the selecting unit 023 refers to a storage apparatus that is not shown with ID information of a patient used as a key, and acquires information related to the selected holding member 002.

Third Modification of Flow

In Step S104 in the flow, it is also preferable to check whether or not the specific light absorbing region 011 is shielded by the masking member 009 after the subject 001 is placed in the selected holding member provided with the masking member 009. As a method for the checking, it is possible to execute the image analysis of the surface information obtained by the optical imaging performed by the region specifying unit 010 in Step S102. In the case where it is determined that the shielding state is not adequate, the region specifying unit 010 selects the optimum holding member 002 again.

Increase of Signal Strength of Masking Member Region

As described above, the masking member 009 has the effect of reducing the artifact caused by the specific light absorbing region 011. However, on the other hand, a light irradiation amount in the region covered by the masking member 009 is reduced as compared with the light irradiation amount in the region without the masking member 009, and hence the intensity of the photoacoustic wave generated in the region covered by the masking member 009 is also reduced. In addition, there are cases where the acoustic wave is blocked or attenuated depending on the material of the masking member 009. With these phenomena, there is a possibility that, in a produced reconstructed image, a reduction in signal strength or S/N in the subject region shielded by the masking member 009 occurs, and image unevenness occurs due to the reduction therein. Hereinbelow, a process for increasing a photoacoustic signal strength value of the shielded region will be described.

Acquisition of Region of Masking Member

Before the execution of an output value increase process of the shielded region, position information of the masking member 009 is acquired and coordinates of a region serving as a process target are checked. As the position information of the masking member 009, it is possible to use the result of the specification of the position and the size of the masking member 009 in Step S105. Specifically, the region specifying unit 010 acquires position information and size information of the masking member 009 by a method similar to that of the selection of the specific light absorbing region 011. The timing of the acquisition may be any timing as long as the timing is after the mounting of the masking member 009.

When the camera 016 acquires the surface image by performing the optical imaging of the holding member 002 including the masking member 009, the region specifying unit 010 determines that an area indicative of a value of not less than a threshold value in the surface image is the masking member 009. The value used as the threshold value in the image processing at this point may be brightness information or color information and, by emitting light having a wavelength that has a large difference between reflection spectrum information of the masking member 009 and the reflection spectrum of the subject 001, and detecting color components, detection accuracy is improved. For example, in the reflection spectrum of the skin, the reflectivity tends to be higher as the wavelength is longer in a visible light region. Accordingly, by using silver or aluminum that reflects a blue component well in the masking member 009, emitting light having the blue component to thereby acquire the image, and detecting the tinge of the blue component, it is possible to detect the masking member 009 with high accuracy.

In addition, by causing the calculated shape of the masking member 009 to correspond to shape information of the masking member 009 selected by the region specifying unit 010 and evaluating the shape thereof, it is possible to improve the detection accuracy. As the correspondence process, it is possible to use pattern matching and area evaluation.

Note that the information related to the region of the masking member 009 may also be acquired from the shape of the masking member 009 selected by the region specifying unit 010 before the imaging instead of acquiring the information related thereto by the optical imaging.

Increase Process

A process for increasing the photoacoustic signal strength of the region covered by the masking member 009 includes a plurality of methods such as (a) a process during the photoacoustic measurement, (b) a signal process that handles a received signal, and (c) an image process that handles an image after reconstruction. Hereinbelow, each process will be described. Note that these processes may be executed independently or a plurality of the processes may be combined.

(a) The Process During the Photoacoustic Measurement

When light is applied to the surrounding part of the masking member 009, as a method for compensating for a reduction in light amount, it is effective to increase the light amount within a range in which the safety of the laser is maintained. With this, it is possible to increase the intensity of the photoacoustic wave. In addition, an increase in the number of times of the light irradiation (an increase in the number of times of the photoacoustic measurement) is also effective. It is possible to improve the S/N ratio by performing multiplication of the obtained signal. In addition, it is also effective to increase a reception gain at the time of reception of the acoustic wave when light is applied to the surrounding part of the masking member 009. With this, it is possible to intensify the signal component deriving from the weak photoacoustic wave caused by the measurement light that propagates to the region shielded by the masking member 009 via the inside of the subject. In other words, when the masking member 009 is included in the irradiation region, the light irradiating unit 004 increases the light amount of the light or the number of times of the light irradiation to a level higher than a level when the masking member 009 is not included in the irradiation region.

In addition, when light is applied to the region other than the surrounding part of the masking member 009, there are cases where each receiving element 025 of the probe 005 receives the acoustic wave that passes through the masking member 009. This phenomenon often occurs in the case where the receiving elements are disposed in a wide area, as in the arc-shaped or cup-shaped probe 005. It is possible to strengthen the signal deriving from the region covered by the masking member 009 also by increasing the gain when such an acoustic wave is received. The target signal of which the gain is increased in this process can be calculated based on the coordinates of the receiving element 025, information related to the arrangement region of the masking member 009, and information related to the light irradiation position.

(b) The Process of the Reception Signal

It is possible to correct unevenness of the signal value caused by blocking of light by increasing the strength of the signal received by the receiving element 025 when light is applied to the surrounding part of the masking member 009. In addition, in the case where the signal of each receiving element that is seen at the time of the reconstruction passes through the masking member 009, for the correction of the unevenness, it is also effective to increase the signal value by a value corresponding to acoustic attenuation at the time of passage through the masking member 009. Note that, in the case where the thickness of the masking member 009 is large, when the sound wave passes through the masking member 009, there are cases where phase shift becomes larger than that when the sound wave dose not pass through the masking member 009. In these cases, it is possible to prevent a reduction in resolution by correcting the phase shift of the signal that passes through the masking member 009.

(c) The Image Process

In the image after the reconstruction, a difference between the output value of the image at the position of the masking member 009 and the output value of the region without the masking member 009 occurs due to the presence of the masking member 009, and brightness unevenness occurs. To cope with this, it is preferable to adjust the output of the image at the position of the masking member 009 such that the output thereof matches that of the image in the region without the masking member 009. The adjustment amount is determined based on acoustic wave transmission characteristics and light transmission characteristics of the masking member 009, a measurement pattern, and a signal processing condition. In addition, it is also effective to compare the output value of the image at the position of the masking member 009 with the output value of the image in the surrounding region and calculate the adjustment amount such that the difference therebetween is reduced. As a result, the brightness unevenness in the image becomes inconspicuous.

As described thus far, according to the method of the present invention, even in the case where the absorber having the large light absorption amount is present on the surface of the subject of the photoacoustic measurement, light to the absorber can be blocked by using the masking member. As a result, the photoacoustic wave generated from the absorber is suppressed, it is possible to acquire excellent characteristic information in which the artifact is reduced, and it becomes possible to produce image data useful for the diagnosis.

### Second Embodiment

In the present embodiment, a description will be given of an adaptive selection method of the masking member 009 and the holding member 002, and a presentation method of the selected holding member 002 in S103 in the flow of the first embodiment. Note that the description of the same processes and configurations as those in the first embodiment will be omitted. Herein, it is assumed that the subject 001 is the breast of the examinee, and the specific light absorbing region 011 is the nipple of the breast. Selection of the holding member adapted to the position and the size of the specific light absorbing region 011 acquired based on the camera image is performed.

Configuration

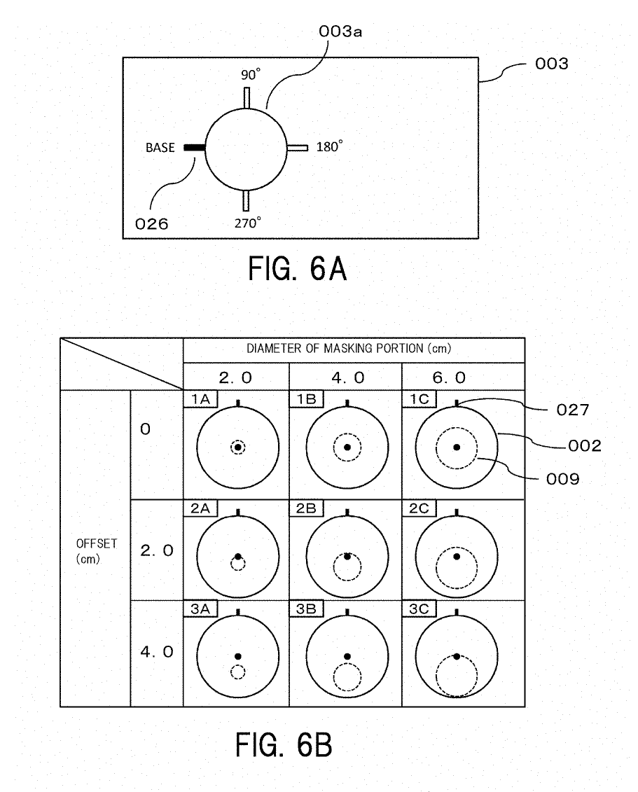

FIG. 6A is a top view showing the supporting base 003 on which the examinee lies facedown and the opening portion 003a. To the opening portion 003a, the cup-shaped holding member 002 for holding the breast is fixed by hooking. In the surrounding part of the circular opening portion 003a of the present embodiment, a plurality of reference tick marks 026 are provided at intervals of 90° with a direction in which the head of the examinee is disposed (the left side in the drawing) used as the basic point (BASE). The reference tick mark 026 may be produced by using paint or stickers, and may also be provided by bonding or burying the reference tick mark 026 to or in the supporting base 003.

FIG. 6B is a conceptual view when the selecting unit 023 selects the holding member 002 adaptively, and is a list of a plurality of the holding members 002 included in the holding member set 2000. Note that, in the drawing, a flange (attaching portion 020) of each holding member 002 is omitted and only a contact portion 021 is shown. In this example, it is assumed that the size and the shape of the holding member 002 are fixed and the holding member 002 has a circular shape having a diameter of 12 cm when viewed from above in a state where the holding member 002 is mounted to the opening portion 003a. The masking member 009 is circular.

The horizontal axis in FIG. 6B indicates the diameter (cm) of the contact portion 021 of the masking member 009, and shows three diameters of 2, 4, and 6 cm. The vertical axis therein indicates an offset (cm) of the center of the masking member 009 relative to the center of the holding member 002. For the convenience of description, IDs of 1A to 3C are provided for each type of each holding member 002 included in the holding member set. Each holding member 002 has amounting tick mark 027 corresponding to the reference tick mark on the side of the opening portion 003a. It is preferable to provide the mounting tick mark 027 in the flange-like attaching portion 020 of the holding member 002 such that the propagation of the acoustic wave and the measurement light is not hindered.

Process Flow

In Step S103 of the flowchart in FIG. 5, the selecting unit 023 acquires size information of the nipple and position information of the nipple in the breast acquired by the region specifying unit 010. The size of the masking member 009 is determined based on the size information such that the masking member 009 covers an area slightly larger than the nipple. In addition, the offset and the mounting angle of the holding member 002 in the supporting base 003 are determined based on the position information.

In the case where the size of the nipple is, e.g., 3.5 cm in diameter, as the masking member, the masking member having a diameter of 4.0 cm is selected. In the case where the center position of the nipple is displaced by 2 cm relative to the center of the holding member 002 to the side of the head of the examinee in a state in which the breast is held by the holding member 002, 2.0 cm is selected as the offset. As a result, the holding member 002 with ID=2B is selected. In addition, as the mounting angle indicative of which reference tick mark 026 on the side of the opening portion the mounting tick mark 027 on the side of the holding member corresponds to, “180°” is selected. The selecting unit 023 presents ID information of the holding member 002 and the mounting angle in the displaying unit 008.

Modification

The selecting unit 023 may adaptively select the size of the holding member based on the size of the breast. In this case, it follows that the conceptual view of FIG. 6B has three axes of the size of the holding member, the size of the masking member, and the offset of the masking member. In this case, although cost for preparing the holding member is required, excellent holding corresponding to the size of the breast is allowed, and it is possible to implement improvements in measurement accuracy and the satisfaction of the examinee.

According to the present embodiment, the selecting unit 023 selects the holding member 002 based on the surface image of the subject from the holding member set 2000 that has a plurality of the holding members each having the masking member 009 of which at least one of the position and the size is specified. At this point, it is possible to adaptively select the holding member 002 provided with the masking member 009 that is appropriate in terms of at least one of the position and the size. Further, as the determined holding member 002, the selecting unit 023 selects the holding member 002 from the holding member set 2000 based on an identifying portion. The identifying portion is a portion capable of determining information that allows identification of at least any of the position, the size, and the shape of the holding member. The identifying portion may be an electronic component such as an IC tag, or may also be a sticker or a label. The identifying portion may be disposed in the holding member included in the holding member set 2000, or may also be associated with the holding member included in the holding member set 2000 and stored. The state in which the identifying portion is associated therewith and stored corresponds to, e.g., a state in which a label on which information indicative of the characteristic of the holding member is described is disposed in a storage container capable of storing all of the holding members included in the holding member set.

The operator can fix the selected holding member 002 to the supporting base 003 at the selected mounting angle of the holding member 002 based on mounting angle information related to the presented ID information. Accuracy in the mounting of the holding member 002 and usability are improved. In the case where the shape of the holding member 002, when viewed in plan view, is circular, rotation when the holding member 002 is mounted to the opening portion 003a is free, and hence the mounting angles are not limited to angles of 90-degree increments. In addition, the present embodiment can also be implemented as the set 2000 of the holding members that include mutually different masking members 009 and are interchangeably arranged in the supporting base 003.

### Third Embodiment

With regard to the photoacoustic apparatus of the present embodiment, a description will be given of an apparatus that detects the position and the size of the specific light absorbing region, and arranges the masking member corresponding to the position and the size thereof. The description of the same processes and configurations as those in the first embodiment will be omitted.

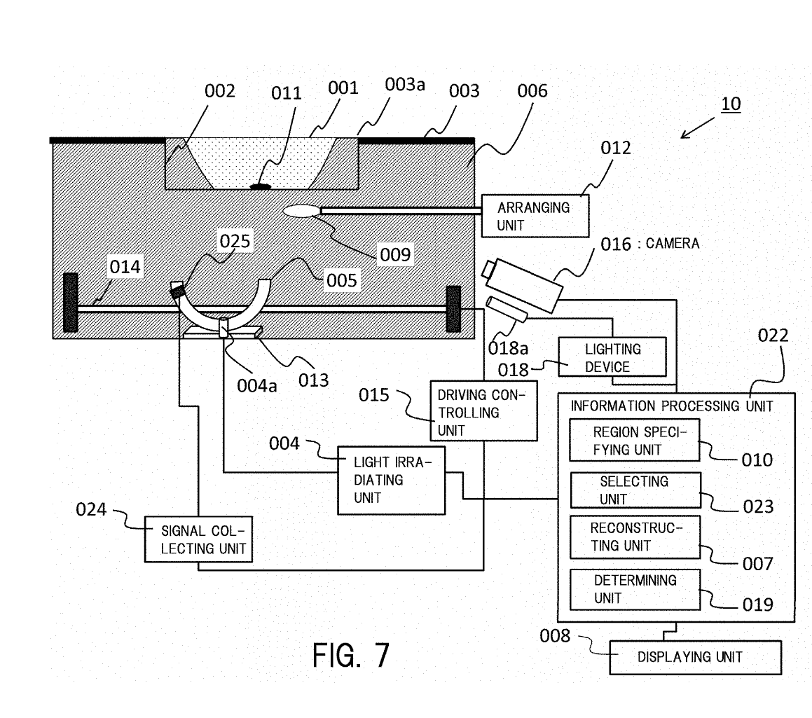

A description will be given of the configuration of the photoacoustic apparatus 10 of the present embodiment by using FIG. 7. In the present embodiment, the selecting unit 023 selects the optimum masking member 009 based on the position and the size of the specific light absorbing region 011 obtained from the region specifying unit 010. The apparatus in the present embodiment includes an arranging unit 012 that arranges the selected masking member 009 in the specific light absorbing region 011.

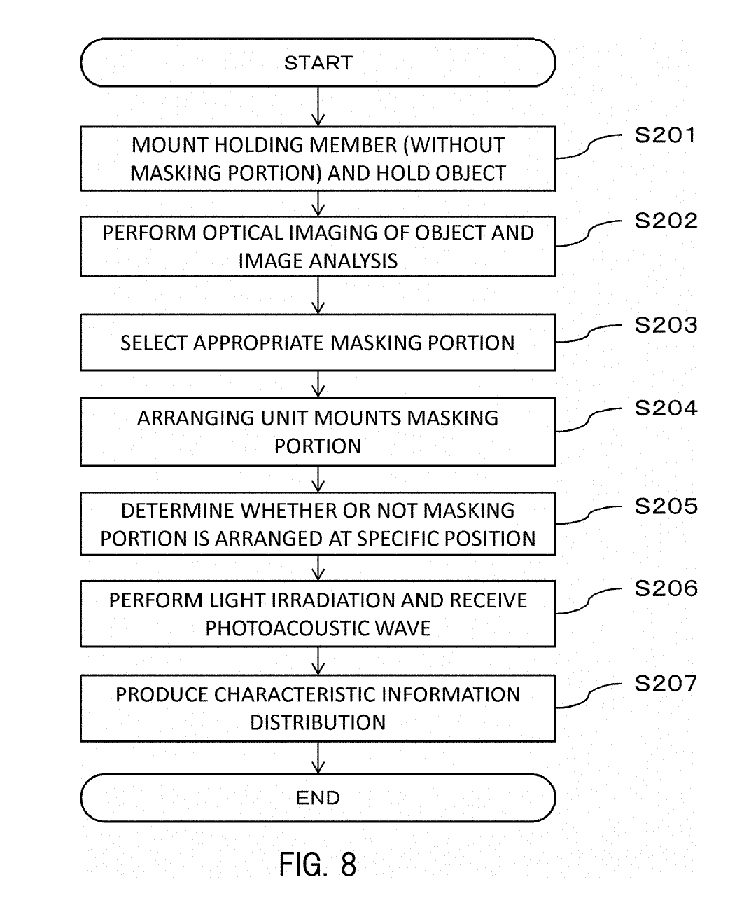

FIG. 8 is a flowchart of the present embodiment. Steps S201 and S202 are the same as those in the first embodiment. In Step S203, the selecting unit 023 selects the masking member 009 adaptively to the size of the specific light absorbing region 011 of the surface of the subject calculated in the region specifying unit 010. Subsequently, in Step S204, the arranging unit 012 mounts the masking member 009 at the position on the holding member corresponding to the specific light absorbing region 011. Next, in Step S205, it is possible to determine whether or not the masking member 009 is arranged at the specific position based on information related to the arrangement operation of the arranging unit 012. Subsequent Steps S206 and S207 are the same as those in the first embodiment.

That is, next, in Step S206, the light irradiation from the irradiation opening 004a of the light irradiating unit 004 is started based on the information that the masking member 009 is arranged at the specific position in Step S205. Subsequently, the receiving element 025 of the probe 005 receives the photoacoustic wave generated from the breast, converts the photoacoustic wave to the electrical signal, and outputs the electrical signal. At this point, by operating the driving mechanism 014 using the driving controlling unit 015, the wide-area measurement is allowed. Next, in Step S207, the image reconstructing unit 007 produces the characteristic information distribution based on the electrical signal. With this, it is possible to acquire the characteristic information distribution indicative of the function information of the inside of the subject.

In the case where the determination result in Step S205 indicates that the masking member 009 is not arranged at the specific position, in Step S206, the photographing operations of the light irradiating unit 004, the probe 005, the driving controlling unit 015, the driving mechanism 014, and the signal collecting unit 024 are stopped.

The element of which the operation is changed in Step S206 based on the determination result in Step S205 is the same as that in the first embodiment.

The masking member 009 in the present embodiment needs strength that prevents the masking member 009 from being damaged even when the masking member 009 is moved in the acoustic matching liquid 006 by the arranging unit 012. For example, as shown in FIGS. 3A and 3B, the masking member 009 obtained by depositing the thin film 009a made of gold on the substrate 009b that is the circular flat PET plate having a thickness of 150 μm can have the required strength.

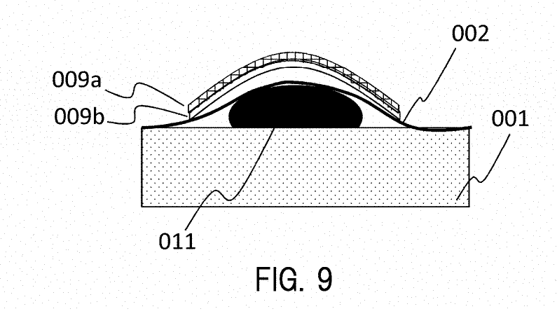

In addition, in the case where the specific light absorbing region 011 has the protruding shape, as shown in FIG. 9, it is appropriate to use the masking member 009 having a shape that secures space in which the specific light absorbing region 011 enters.

Note that, in the present embodiment, there is a distance between the masking member 009 and the holding member 002. Accordingly, when the protrusion amount 009p of the masking member from the specific light absorbing region 011 is determined, it is necessary to consider a bending-around amount of light corresponding to the distance.

In the case of the present embodiment as well, in consideration of the influence by the movement and displacement after the placement of the subject 001, it is effective to use the relatively large masking member 009 and perform the mounting of the masking member in which the displacement amount and the displacement direction are considered.

In addition, also in the present embodiment, the displacement having the displacement amount larger than the assumed displacement amount can occur. To cope with this, it is effective to detect the specific light absorbing region 011 using the region specifying unit 010 again after the mounting of the masking member 009. With this, it is possible to check whether or not the specific light absorbing region 011 is shielded by the masking member 009. The timing of the checking is preferably immediately before the measurement or after the end of the measurement. In the case where the specific light absorbing region 011 is determined at the time of the checking immediately before the measurement, the arranging unit 012 mounts the masking member 009 again. In the case where the specific light absorbing region 011 is determined after the end of the measurement, a message that urges the operator to perform re-measurement is issued or a message indicating that the specific light absorbing region 011 is determined is presented. In this case, it is preferable to present the shielding state to the operator such that the operator can determine whether or not the re-measurement is executed. That is, the region specifying unit 010 is configured to be capable of infrared imaging of the subject 001 in the wavelength range including infrared light or near-infrared light, and the region specifying unit 010 can be configured to also serve as the determining unit.

In the present embodiment as well, it is preferable to increase the strength of the photoacoustic signal deriving from the region in which light is blocked by the masking member 009. In this increase process, it is possible to determine the position and the size of the masking member based on the coordinate information when the arranging unit 012 has mounted the masking member 009. The signal collecting unit 024 and the information processing unit 022 perform the increase of the reception gain, the reception signal process, or the image process such that the signal strength of the shielded region is increased.

Shape of Masking Member and Arrangement Method

The arranging unit 012 includes an arm for mounting the masking member 009 at the position of the holding member 002 corresponding to the specific light absorbing region 011. The arm is configured to hold the masking member 009 at the tip thereof, and be driven by machine control. The arm extends the tip to a place where a set 9000 of the masking members 009 is stored and holds the specific masking member 009, and then moves the tip to a position in the vicinity of the surface of the holding member 002, and fixes the masking member by bonding or the like. Note that, when the region specifying unit 010 specifies the masking member 009, the operator informed of the information in the displaying unit or the like may cause the tip of the arm to hold the masking member 009.

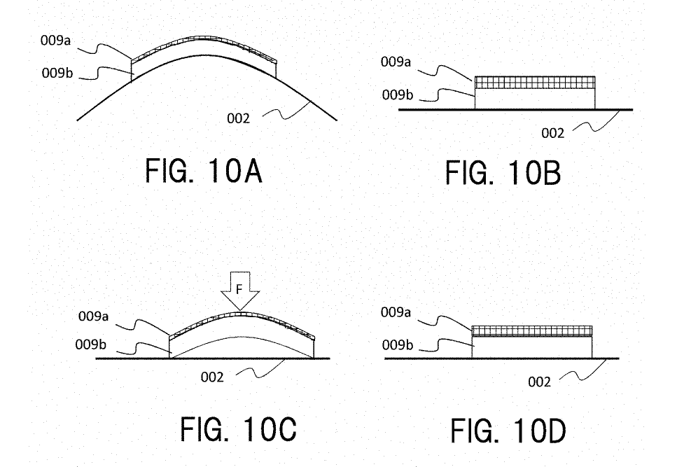

FIGS. 10A to 10D show the state of the bonding. In the case where the shape of the holding member 002 is stabilized (e.g., in the case where the holding member 002 is formed of a resin material such as PET), a resin material or a metal plate of which the shape is stabilized is suitable also for the masking member 009. FIGS. 10A and 10B show the holding member 002 that maintains a curved shape and the holding member 002 that maintains a flat shape, respectively. In this case, as the shape of the masking member 009, the shape that extends along the holding member 002 is preferable. In the case where the surface of the holding member 002 is curved, the surface of the masking member 009 is caused to match the curve. The arranging unit 012 presses the masking member 009 against the holding member 002 and pushes the acoustic matching liquid 006 between the masking member 009 and the holding member 002 away to thereby increase airtightness between the holding member 002 and the masking member 009 and bond them together.

In the case where a material that bends flexibly such as gel or rubber is used as the substrate 009b of the masking member 009, it is preferable to form, of both surfaces of the masking member 009, the surface on the side that comes into contact with the holding member 002 into a sucker-like shape. FIG. 10C shows the state of the masking member 009 before the bonding, and FIG. 10D shows the state thereof after the bonding. When the arranging unit 012 presses the masking member 009 against the holding member 002 with a force Fin the state of FIG. 10C, as shown in FIG. 10D, the acoustic matching liquid 006 between the masking member 009 and the holding member 002 is pushed away, the airtightness is increased, and an adhesive force is increased.

There are cases where it is not possible to stably bond the masking member 009 depending on the material and the shape of the holding member 002. In these cases, the masking member 009 may be held by pressing and fixing by holding the masking member 009 between the arm and the holding member 002. However, in this case, there is a possibility that the arm blocks the irradiation light or the acoustic wave to cause the artifact, or image degradation is caused by blocking the acoustic wave from the subject 001. To cope with this, it is appropriate to use the arm that has a slim shape and high stiffness, and does not absorb the acoustic wave. For example, the arm obtained by depositing gold on the surface of metal such as stainless is used.

According to the present embodiment, even in the case where the absorber having the large light absorption amount is present on the surface of the subject of the photoacoustic measurement, the appropriate masking member is selected by the selecting unit. By mounting the masking member to the holding member using the arranging unit, light to the absorber at the time of the photoacoustic measurement is blocked, and it becomes possible to acquire the excellent photoacoustic image in which the artifact is reduced.

### Fourth Embodiment

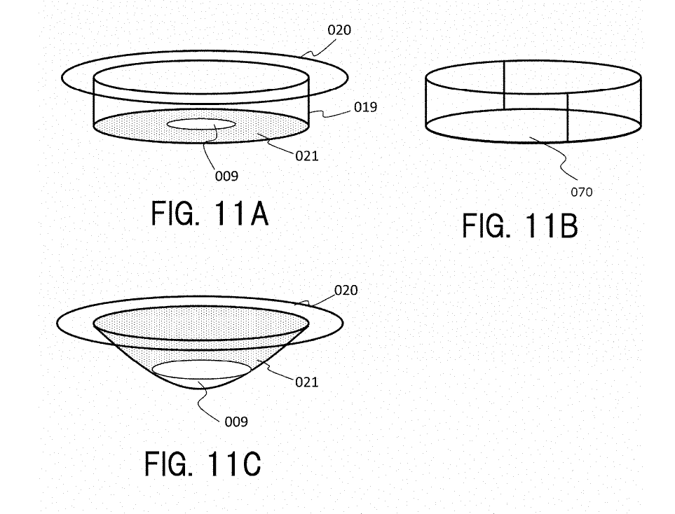

In the present embodiment, various forms of the holding member 002 will be described with reference to FIGS. 11A to 11C. The same components as those in the above embodiments are designated by the same reference numerals and the detailed description thereof will be omitted. The contact portion 021 is a part of the holding member 002 that comes into contact with the subject 001. The masking member 009 described in each embodiment described above is provided on the contact portion 021. The holding member 002 shown in FIG. 11A includes a cylindrical portion 070, the flange-like attaching portion 020 that is attached to the opening portion 003a of the supporting base 003, and the contact portion 021 that is disposed on the opposite side of the attaching portion 020 with the cylindrical portion 070 interposed therebetween.

As the material of the cylindrical portion 070, transparent resins such as acrylic, polycarbonate, and PET are preferable. The same materials as those of the cylindrical portion 070 can be used for the contact portion 021, and the thickness of the contact portion 021 is preferably smaller than that of the cylindrical portion 070. The same materials as those in the first embodiment described above can be used for the masking member 009. As the masking member 009, for example, metal deposited on the surface of the contact portion 021 and a metal plate stuck to the surface thereof are preferable. In the case where a scatterer is used as the masking member 009, it is possible to apply a scattering material to the surface of the contact portion 021 or stick a member to which the scatterer is added to the surface thereof. In the present embodiment, the contact portion 021 that is made of transparent PET and has a thickness of 0.1 mm is bonded to the cylindrical portion 070 that is made of transparent acrylic and has a thickness of 3 mm, and the masking member 009 is formed by depositing gold on the central part of the contact portion 021.

Note that a distance between the masking member 009 and the specific light absorbing region 011 is reduced by depositing, sticking, or applying the masking member 009 to the surface of the contact portion 021 on the side of the subject 001, and hence it is possible to suppress the irradiation light to the specific light absorbing region 011 resulting from diffraction. Note that, in the case where the contact portion 021 is extremely thin, the influence of the diffracted light is little even when the masking member 009 is formed on a surface that does not come into contact with the subject 001. Thus, by forming the masking member 009 on the surface of the contact portion 021 that does not come into contact with the subject 001, the degradation of the masking member 009 caused by the contact with the subject 001 is prevented. In the present embodiment, the contact portion 021 is sufficiently thin, and hence gold is deposited on the surface that does not come into contact with the subject 001.

In the case where gold is deposited on a PET film, when Cr is deposited between PET and gold, the adhesive property of gold is increased. However, there is a possibility that reflected light from the side of the subject 001 is applied to the Cr surface and the acoustic wave serving as the factor of the artifact is generated. To cope with this, it is effective to deposit the masking members 009 on both surfaces of the contact portion 021.

As the material of the contact portion 021, it is also possible to use a sheet material such as a rubber sheet or a mesh sheet. As the cylindrical portion 070, as shown in FIG. 11B, it is also possible to use the cylindrical portion having a beam structure. Such a structure is effective in that the acoustic wave is caused to propagate to the receiving element smoothly. In addition, as shown in FIG. 11C, it is also possible to use a configuration in which the cylindrical portion 070 is not provided and the contact portion 021 is mounted to the attaching portion 020. In the case of this configuration, it is possible to provide the cup-shaped holding member 002 having a curve extending along the shape of the breast. In the case where the subject 001 is the breast, the nipple or the areola is the specific light absorbing region 011. The nipple or the areola is often located at the center of the holding member 002, and hence it is preferable to mount the masking member 009 to the center of the contact portion 021.

### Fifth Embodiment

In the first embodiment, the region specifying unit 010 acquires the information related to the specific light absorbing region 011, and the selecting unit 023 selects the appropriate holding member 002 and presents the selected holding member 002 to the operator. In the third embodiment, the arranging unit 012 mounts the masking member 009 to the holding member 002 based on the information related to the specific light absorbing region 011. In the present embodiment, the information processing unit 022 determines the position at which the masking member 009 is mounted to the holding member 002 based on the information related to the specific light absorbing region 011 determined by the region specifying unit 010, and presents the position to the operator. Hereinbelow, the description of the same configurations as those in each embodiment will be omitted, and parts peculiar to the present embodiment will be described.

The system configuration of the present embodiment is substantially the same as that in the first embodiment, but a mounting position specifying unit is provided in the information processing unit 022 instead of the selecting unit 023. In the present embodiment, it is assumed that the set 9000 of a plurality of the masking members 009 having mutually different sizes is prepared in advance. The mounting position specifying unit selects the masking member 009 capable of shielding the specific light absorbing region 011 from the irradiation light from the mask portion set 9000 including a plurality of the masking members that is prepared in advance based on the position, the size, and the shape of the specific light absorbing region 011. At this point, in order to reduce the influence of the diffracted light, it is appropriate to select the masking member 009 having the size that allows the masking member 009 to slightly protrude from the specific light absorbing region 011.

Subsequently, the mounting position specifying unit presents a specific position at which the masking member 009 is to be mounted to the operator via the user interface such as the displaying unit 008. The presentation method is not particularly limited and, for example, as in the second embodiment, the specific position may be specified by the offset from the center of the holding member 002 and the angle from a cephalocaudal direction. In addition, the holding member 002 may be divided into virtual meshes on the displaying unit 008, and the mesh in which the masking member 009 is mounted may be specified. The mounting method of the masking member 009 is not particularly limited, and the mounting method includes a method in which that the masking member 009 is stuck to the holding member 002 using the sucker-like shape similarly to the third embodiment, and a method in which an adhesive material is applied to the substrate 009b.

In the case of the present embodiment, the masking member 009 is mounted by manipulation of the operator, and hence it is preferable to perform the optical imaging by the camera 016 and the image analysis by the region specifying unit to check the mounting position again after the masking member 009 is mounted. In the case where positional displacement between the specific light absorbing region 011 and the masking potion 009 is large, the displaying unit 008 presents a correction method of the position. According to the present embodiment, the size and the mounting position of the masking member 009 are determined adaptively to the position and the size of the specific light absorbing region 011, and hence it is possible to produce the photoacoustic image in which the artifact is reduced.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-245348, filed on Dec. 19, 2016, and, Japanese Patent Application No. 2017-225660, filed on Nov. 24, 2017, which are hereby incorporated by reference herein in their entirety.

What is claimed is:

1. A photoacoustic apparatus comprising:
a light irradiating unit configured to irradiate a subject that is part of an examinee with light;
a probe configured to receive an acoustic wave generated from the subject irradiated with the light and output a signal; and
a determining unit configured to determine, based on information related to a specific light absorbing region on a surface of the subject, whether or not a masking member that optically masks the specific light absorbing region is arranged at a specific position.

2. The photoacoustic apparatus according to claim 1, wherein
the information is related to a position or a size of the specific light absorbing region.

3. The photoacoustic apparatus according to claim 1, wherein
the specific position is located between the specific light absorbing region and the light irradiating unit.

4. The photoacoustic apparatus according to claim 1, further comprising;
a supporting base that supports the examinee and is provided with an insertion opening through which the subject is inserted and an installation portion around the insertion opening configure to be installed a holding member for holding the subject at a position overlapping the insertion opening.

5. The photoacoustic apparatus according to claim 4, wherein
the determining unit is configured to determine whether or not the holding member has the masking member at the specific position.

6. The photoacoustic apparatus according to claim 1, wherein
photoacoustic measurement is started based on a determination result of the determining unit.

7. The photoacoustic apparatus according to claim 4, further comprising:
a holding member selecting unit configured to select the holding member, provided with the masking member based on information related to a position or a size of the specific light absorbing region, from a holding member set including a plurality of holding members in which the masking members are arranged such that at least one of a position and a size of the masking member differs, from one holding member to another.

8. The photoacoustic apparatus according to claim 1, further comprising:
a region specifying unit configured to acquire information related to a position and a size of the specific light absorbing region.

9. The photoacoustic apparatus according to claim 1, further comprising:
an image reconstructing unit configured to image the signal; and
a displaying unit configured to display a photoacoustic image outputted from the image reconstructing unit.

10. The photoacoustic apparatus according to claim 4, further comprising:
an arranging unit configured to arrange the masking member at a position of the holding member corresponding to the specific light absorbing region, based on the information related to the specific light absorbing region on the surface of the subject.

11. The photoacoustic apparatus according to claim 1, wherein
the specific light absorbing region is a region, in which absorbance is relatively high, on the surface of the subject.

12. The photoacoustic apparatus according to claim 1, wherein
the masking member is configured to scatter or reflect at least part of the light emitted from the light irradiating unit.

13. The photoacoustic apparatus according to claim 1, wherein,
when spectral transmittance and spectral reflectivity corresponding to a center wavelength λ of the light emitted from the light irradiating unit are T(λ) and R(λ) respectively, the masking member satisfies the following general expression (1):
1−(T(λ)+R(λ)<T(λ)+R(λ) (1).

14. The photoacoustic apparatus according to claim 13, wherein
the spectral transmittance T(λ) and the spectral reflectivity R(λ) satisfy the following general expression (2):
T(λ)<R(λ) (2).

15. The photoacoustic apparatus according to claim 13, wherein
the spectral transmittance T(λ) and the spectral reflectivity R(λ) satisfy the following general expression (3):
1≥T(λ)+R(λ)≥0.75 (3).

16. The photoacoustic apparatus according to claim 8, wherein
the region specifying unit is configured to double the determining unit, and
the region specifying unit is configured to determine whether or not the masking member is arranged at the specific position.

17. The photoacoustic apparatus according to claim 1, wherein
the light irradiating unit is configured to increase, when the masking member is included in an irradiation region, a light amount of the light or the number of times of irradiation of the light to a level higher than a level when the masking member is not included in the irradiation region.

18. The photoacoustic apparatus according to claim 1, wherein
the determining unit is configured to output information indicating whether or not the masking member is used, based on an input command of an operator.

19. A holding member set, including a plurality of holding members which holds a subject and is selectively used for a photoacoustic apparatus, the photoacoustic apparatus receives and images photoacoustic waves generated from the subject held by the holding member, wherein
the holding member set is configured to have the plurality of holding members in which masking members that are different in at least any of position, size, and shape are arranged correspondingly to a specific light absorbing region on a surface of the subject.

20. A masking member set, including a plurality of masking members which is to be arranged on a specific position of a holding member and is selectively used for a photoacoustic apparatus, the photoacoustic apparatus receives and images photoacoustic waves generated from a subject held by the holding member, wherein
the masking member set includes the plurality of masking members that are different in at least one of shape and size from each other.
